(19) United States
(12) Patent Application Publication
(10) Pub. No.: US 2005/0277158 A1
(43) Pub. Date: Dec. 15, 2005

(54) Method and kit for donor specific complement-fixing antibodies crossmatch

(76) Inventor: Ge Chen, Los Angeles, CA (US)

Correspondence Address: MARK K JOHNSON, 20925 CROSSROADS CIRCLE, WAUKESHA, WI, 53186, US

(21) Appl. No.: 11/115,442

(22) Filed: Apr. 27, 2005

Related U.S. Application Data
(60) Provisional application No. 60/578,354, filed on Jun. 10, 2004.

(51) Int. Cl. G01N 33/53; G01N 33/567; G01N 33/555

(52) U.S. Cl. 435/7.2

(57) ABSTRACT

Provided is a flow cytometry method and kit composition for semi-quantitatively determining complement fixing antibodies (CFAbs). The said antigen specific CFAbs in the sample will react with the antigens on the solid carriers or the surfaces of target cells as well as the labeled complement or labeled anti-complement antibody at the same time; and the labeled target cell specific antibody will bind to the target cell surfaces. Then the sample is analyzed to determine the amount of complements fixed by CFAbs, especially the amount of complements fixed by CFAbs on the surfaces of particular target cells or donor specific HLA antigens pre-captured by corresponding antibodies fixed on solid microparticles for evaluating whether there is CFAbs and their relative concentration.

This specification is related to provisional application No. 60/578,354 filed on Jun. 10, 2004.

### TECHNICAL FIELD

The invention relates to a method for complement-fixing antibodies (CFAbs) measurement. Complement-fixing antibodies are directly or indirectly measured by detecting the complements fixed by CFAbs in an antigen-antibody reaction system. The method of the invention is particularly suitable for cross-match in organ transplantation.

### TECHNICAL BACKGROUND

Rejection in organ transplantation is the immunological responses to the allogeneic graft (non-self) by the recipient's immune system. When the proteins on the surface of graft (non-self antigens) contact the immune system of the recipient, the non-self antigens are immunologically recognized. This recognition sequentially activates both the humoral and cellular immune systems of the recipient to attack and destroy the graft and result in organ transplantation failure.

In the graft rejection of organ transplantation, the recipient's immune system recognizes both the MHC (Major histocompatibility complex) phenotype and the short peptide complex of the graft through MHC recognition mechanism. For human being, MHC is referred to as human lymphocyte antigens (HLA). If the HLA of the donor doesn't match that of the recipient, the graft is recognized to be non-self and is immunologically attacked by the recipient's immune system, which causes “graft rejection”.

Hyperacute rejection is mainly mediated by the recipient's humoral immunity components, i.e. pre-existing antibodies against unmatched donor's HLA molecules in the recipient. These antibodies arise from the exposure of the recipient's immune system to non-self HLA molecules during the multi-pregnancies, blood-transfusion, previous transplantation etc. In a hyperacute rejection, recipient's HLA antibodies can rapidly recognize and bind to donor's HLA molecules and induce the complement-dependent cytotoxicity (CDC) effects by fixing complements and elicit complement cascade to damage graft cells. A hyperacute rejection happens almost immediately, usually from a few minutes to several hours. Both of humoral immunity and cellular immunity are involved in acute and chronic graft rejections through a complicated immunology mechanism. In order to prevent the graft rejection in organ transplantation, besides HLA types matching between a donor and a recipient, crossmatch is considered as a most important test to prevent the hyperacute rejection. Generally, crossmatch is performed between the donor's lymphocytes and the recipient's serum to determine whether there are antibodies against the donor's HLA in the recipient. After transplantation, in order to monitor the occurrence of acute and chronic rejections as well as predict survival time of a graft, it is necessary to perform the test dynamically for monitoring the occurrence and level of changes of HLA antibodies in a recipient. Crossmatch and PRA (panel reactive antibodies) are most commonly used tests in clinical transplantation practices.

Human HLA antibodies can be classified into two categories according to whether the antibody can fix and activate complement to induce CDC effects or not. One category is referred to as complement-dependent cytotoxic antibodies (CDC-Abs) or complement fixing antibodies (CFAbs), which can fix complements and activate the complement system to induce CDC effects after binding to corresponding HLA molecules, which is the main cause for a hyperacute graft rejection. Its clinical significance in the induction of immunological rejection is well known. These antibodies are the humoral immunity components that directly lead to immunological graft rejection, especially the hyperacute rejection. The other category of antibodies is referred to as non-complement fixing antibodies (Non-CFAbs). Although they are able to specifically bind to HLA molecules, they neither fix complements nor induce CDC effects. This category of antibodies exist in a natural manner in human body even under the condition that the immunological system has never contacted with any non-self HLA antigens. They bind to autologous or allogeneic HLA molecules without activating complement and inducing CDC effects. The function of this category of antibodies is not very clear so far. However, most researchers are of the view that these antibodies are part of natural antibodies and have benefit effects for maintaining normal health and immunity homeostasis of human being and other organisms. In-depth studies have been conducted on it in the United States and France. The results from numerous in vitro and in vivo studies and clinic researches have been showing the same conclusion that the natural antibodies exist in normal human body, which, due to their effects of immunological regulation and protective effects with respect to maintaining the health of living organisms and these natural antibodies can be effectively used as an immunosuppressive therapy of the graft rejection in transplantation clinical practices.

At present, the methods for determining HLA antibodies are classified into two categories. One is CDC serological method, named Terasaki's microlymphocytotoxicity test (Patel R, Terasaki P I., N Engl J Med 1969; 280:735-9), which is generally accepted as a standard method for determining CDC effects caused by CFAbs. In this method, CDC-induced death rate of lymphocytes is scored and gives a positive or negative judgment. As a standard method for measuring CDC effects, it is characterized by directly determining the final effect of CDC induced by CFAbs—cells death. It is a method of determining the biological effect of CFAbs. This method has been widely used in crossmatch of clinical transplantation and PRA assay for more than thirty years. Since the cells death rate is estimated by manually counting dead cells under a microscope, there are often differences from a same sample between different operating persons. Besides, it has other disadvantages like time-consuming, low sensitivity, technical complexity, allogeneic complements use, and many other uncontrollable test conditions. The other methods can only detect total antibodies that bound to HLA antigens including both CFAbs and non-CFAbs. Flow cytometry crossmatch (IgG-FXM) (Garovoy M R, et al., Transplant Proc 1983; 15:1939-41) has been established to determine the IgG antibodies bound on cell surface. Other alternative methods for the same principle include flow cytometry microspheres IgG assay and ELISA by coating HLA antigens onto either microspheres or microplate wells. The major disadvantage of this category of methods is that the existence of natural HLA antibodies (Non-CFAbs) have been neglected. Non-CFAbs do not induce CDC effects, on the contrary, they eventually could occupy the corresponding HLA antigen binding sites and prevent cytotoxic HLA antibodies (CFAbs) from binding to HLA molecules and block the subsequent CDC effects. Therefore, the above-mentioned methods have obvious disadvantages in terms of principles of methodology, and can't differentiate the above two categories of antibodies that have totally different biological functions. This is the reason why many laboratories, using this category of methods, obtained results inconsistent with those of CDC serological method, or even completely contrary to the clinical outcomes. In the case of intravenous injection of normal human immunoglobulin gamma (IVIG) treating an organ transplantation recipient, the results obtained by these methods are constantly positive, while the patient's clinical condition is normal or improving. In clinical organ transplantation, the results obtained by these methods are often showing false positives, which mislead the selection of donors and result in exacerbation or death of certain patients in urgent need of graft due to losing the chance of transplantation.

A parallel study of cross-match by flow cytometry and traditional serological test was made in 1996 by Christiaans on 190 transplantation patients (Christiaans M H, et al., Transplantation 1996; 15:1341-1347). The results showed that, after transplantation, there was no statistic difference in rejection between the patients who were positive and those who were negative in flow cytometry crossmatch; 28% of the positive patients had improving clinical outcomes. The conclusion was that flow cytometry cross-matching was not superior to the traditional serological cross-matching. A similar conclusion was made by the study of Karuppan (Karuppan s s, et al., Transplantation 1992; 53:666-673).

The study of Chia and Terasaki (Chia D, et al., Tissue Antigens 1991; 37:49-55) in 1991 showed that immunoglobulin IgG from normal person can bind to HLA molecules through the non-hypervariable regions, and the binding can be blocked by specific antiserum of HLA. Using different experimental designs and methodology, we have further proved that normal human IgG can bind with human lymphocytes and almost totally block the cytotoxic effects of specific HLA antibodies. The above studies show that non-CFAbs are also detected in flow cytometry crossmatching which leads to false positive results, and that non-CFAbs are not closely correlated to clinical outcome or even contrary to the same.

Koba (Koka P., Transplantation 1993; 56:207-211) and Kerman (Kerman R H., Transplantation 1999; 68(12):1855-1858) in 1993 and 1999 respectively reported the clinical significance of IgA and IgM HLA antibodies, and the correlation between them and occurrence of rejection. However, these two types of antibodies can't be detected by routine flow cytometry cross-match.

A clinical study was conducted by Dolly et al about suppression of transplantation rejection by IVIG in 1994 (Tyan et al. Transplantation 1994; 57:553-562). The study has proved that IVIG has excellent immune suppression effect on the graft rejection. In vitro studies also proved that IVIG could dramatically inhibit the CDC effects of the sera from high PRA recipients. Further studies on the mechanism (not published) proved that IVIG induced the inhibition effects on CFAbs through binding with lymphocytes. However, IVIG binding is detectable by flow cytometry IgG crossmatch and gives a false positive crossmatch. In this case, a positive crossmatch is often contrary to clinical outcome in the patient who was under the IVIG treatment, and may mislead physicians in connection with the real imunological state of the patients and therefore immunosuppressive treatment.

Another significant disadvantage of the existing methods for determining HLA antibodies is time-consuming.

To sum up, before transplantation, crossmatch for determining the existing CFAbs in the recipient is critical for the success of a graft transplantation. It is also an important index for monitoring graft rejection and predicting the clinical outcome of the recipient after transplantation.

All of the crossmatch methods that are currently used in transplantation practice are not perfectly meet the demands mentioned above. Therefore, there is an urgent need for developing an easy, rapid, accurate, sensitive, specific, stable and standardized crossmatch method in graft transplantation.

### SUMMARY OF THE INVENTION

According to the basic principles of CDC effects, the present invention establishes a method based upon immunological methodology for evaluating CDC effects by detecting the quantity of the complements fixed by CFAbs. During the initial stage of CDC effect, CFAbs bind with corresponding HLA antigens on cell surface and fix complements, the quantity of complements fixed by CFAbs has directly correlated to the strength of CDC effect (cells death). Therefore, CDC effects can be directly evaluated by determining the quantity of complements fixed by CFAbs. On the other hand, labeled antibodies against the characteristic antigens on cell surface can be introduced into the same reaction system of the method to simultaneousely identify CDC effects on different particular cell populations. Consequently, the method can selectively determine CDC effects on different cell populations in a same tube.

The CFAbs crossmatch method of the present invention has significant advantages over existing crossmatching methods. It is theoretically more rationale and the results in actual crossmatch tests are completely consistent with the the clinical overcomes.

The present invention provides a method of crossmatch by determining complement-fixing antibodies, comprising a step of contacting antigens from the donor with a sample from the recipient probably containing complement-fixing antibodies against the antigens and related complements, a step of contacting a labeled complement or labeled anti-complement antibody, a step of removing the non-bound labels from the reaction system, and a step of detecting the signals of the label or reaction signals to determine the existence of said complement-fixing antibodies in the sample and/or concentration thereof; wherein said antigens from the donor are donor's whole nucleate cells or HLA molecules pre-captured by anti-HLA antibodies on the surfaces of solid carriers. Preferably, said label is a fluorescence label, and the fluorescence intensity of the reaction product is determined by a flow cytometer.

Preferably, said antigen is derived from a graft donor (hereinafter referred to as the donor), said sample is derived from a graft recipient (hereinafter referred to as the recipient), said method is a method of crossmatch. The crossmatch method comprises the following steps:

(1) a certain amount of mononuclear cells or WBC from the donor and a certain amount of serum from the recipient are added into a test tube;

(2) after 10 to 120 minutes of incubation, the cells are washed with a washing solution;

(3) the cells are resuspended in a buffer containing at least one fluorescence-labeled antibody against a complement (for example FITC-antibody against human complement C1q/C3), and the cells are washed after 10 to 120 minutes of incubation;

(4) the cells are suspended in a buffer, the fluorescence intensity on cell surface is measured and analyzed by a flow cytometer, and compared with the control of normal male AB serum sample, giving the result of a semi-quantitative crossmatch.

Preferably, the present invention relates to a crossmatch method of T and/or B lymphocytes, which comprises the following steps:

(1) a certain amount of mononuclear cells or WBC from the donor and a certain amount of serum from the recipient are added into a test tube;

(2) the cells are washed with a washing solution after 10 to 120 minutes of incubation;

(3) the cells are resuspended in a buffer containing at least one fluorescence-labeled antibody against a complement (for example FITC-antibody against C1q/C3);

(4) at least one antibody labeled with another fluorescence and directed against a protein on cell surface (for example PE-antibody against CD3, PerCP-antibody against CD19 or CD20) is added;

(5) the cells are washed after 10 to 120 minutes of incubation;

(6) the cells are resuspended in a buffer, the fluorescence intensity of the anti-complement antibody on the surface of T/B cell population (PE-CD3+/PerCP-CD19+ or CD20+) is analyzed by a flow cytometer and compared with the control of normal male AB serum sample, giving the result of a semi-quantitative crossmatching of T/B cell population.

According to another aspect, the present invention relates to a kit for implementing the above method of the invention, which comprises at least containers containing respectively a fluorescence-labeled complement or fluorescence-labeled anti-complement antibody and suitable buffer. In addition, the kit of the invention may optionally contain a fluorescence-labeled antibody against an antigen on the surface of target cells to identify the cell populations, or anti-HLA class I or class II antibodies or a mixture of above two antibodies. In a preferred embodiment of the kit of the present invention, said anti-HLA antibodies are conjugated or coated on solid carriers, therefore being able to capture corresponding HLA antigens from the donor's cell lysates.

### DETAILED DESCRIPTION OF THE INVENTION

The present invention relates to a method for determining complement-fixing antibodies, which comprises a step of contacting an antigen with a sample probably containing complement-fixing antibodies against the antigen and related complements, a step of contacting a labeled complement or labeled anti-complement antibody, a step of removing the unbound label from the reaction system, and a step of detecting the signal of the label or a reaction signal to determine the presence of said complement-fixing antibodies of interest in the sample and/or the concentration thereof. Preferably, said label is fluorescence labels, and the fluorescence intensity is determined by flow cytometry.

In the method of the present invention, said antigens can be any biological antigen capable of binding with complement-fixing antibodies. Said biological antigen can be a target cell with the antigen on its surface or can be a purified antigen. Said biological antigen can be an antigen of HLA class I and/or HLA class II, a target cell with the antigen of HLA class I and/or HLA class II, or a purified antigen of HLA class I and/or HLA class II. Said target cell can be any kind of biological cells of the donor, especially nucleate cells such as lymphocytes, polymophonuclear cells, monocytes, platelets, NK cells, and culture cell lines. In one embodiment of the invention, preferably said cells are T lymphocytes and/or B lymphocytes. Above mentioned cells can be prepared from any sources such as blood, other biological fluids, spleen, lymphonodes, and cultured cells.

It is well known that HLA antigens can be classified into HLA-A, HLA-B, HLA-C, HLA-D (DR, DQ, DP) and some other subtypes. Any subtype or the mixture of the subtypes (pooled HLA antigens) can be used in the method of the invention. In the method of the invention, preferably said antigens are derived from the donor. In a preferred embodiment of the invention, said antigens are fixed on solid carriers.

In the method of the invention, the sample to be detected can be body fluids derived from a human being or an animal. Said body fluids can be, for example, serum, plasma, cerebrospinal fluid, spinal fluid, amniotic fluid, salvia or urine etc. In the method of the invention, said sample is preferably derived from the recipient.

In the method of the invention, said complements can be any complement components, which are involved in complement fixation of antibody and subsequent complement-activated enzymatic chain reaction, or any proteins or other factors regulating and controlling complement activation, which include any components involved in classical or alternative pathway of complement activation. That is, the “complements” of the invention are not limited to the inherent complement components as commonly recognized. To be specific, said complement in the invention can be any of the following: C1(C1q, C1r, C1s), C2, C3, C4, C5, C6, C7, C8, C9; C1qrs, C1qrs, C2a, C2b, C3a, C3b, C4a, C4b, C4b2, C4d, iC3b, C4b2b, C4b2b3b, C3bBb, C3bnBb, C5a, C5b, C5b67, C5b˜8, C5b˜9; C1-inhibitory factor, C4-binding protein, D factor, B factor, P factor (properdin), I-factor, H-factor, S-protein; Ba, Bb, MBP, MCP, DAF(CD55), CR1, CR2, CR3, CR4, CR5, C3aR, C2aR, C4aR, C1qR, CD59. In an embodiment of the invention, said complement is derived from the recipient.

In a preferred embodiment of the invention, said complement is C1q or C3.

In an embodiment of the invention, a fluorescence-labeled complement is used. Said labeled complement can be derived form human being, animal, chemical synthesis or genetic engineering. In this embodiment, the corresponding complement probably contained in the sample should be inactivated.

In another embodiment of the invention, a fluorescence-labeled anti-complement antibody is used. Said anti-complement antibody can be derived from any source and be any type of antibody, which can specifically bind with a complement to be tested. For example, the anti-complement antibody can be a polyclonal antibody, monoclonal antibody, chimeric antibody, single chain antibody or fragment of antibody.

In a preferred embodiment of the invention, it is preferred to add a fluorescence-labeled antibody against a particular antigen on the surface of target cells in order to identify the particular cell population. For example, said antibody against a particular antigen on the surface of target cell is directed against CD antigens of T lymphocytes (CD2 or CD3) or B lymphocytes (CD19 or CD20). The fluorescence label of the antibody against an antigen on the surface of target cell is different from that of the complement/or anti-complement antibody so that the signals of the different fluorescence labels can be identified simultaneously in the same reaction by a flow cytometer.

In an embodiment of the invention, said antibodies against the particular antigens, preferably HLA antigens, are fixed on surfaces of solid carriers. The said anti-HLA antibody molecules can specifically bind to the constant region (or common regions, or public regions) of HLA molecules. When the HLA antibodies fixed on the surface of solid carries are incubated with the cell lysates, the fixed HLA antibodies will capture HLA molecules of the lysates by binding to the constant regions of HLA molecules to leave the rest HLA polymorphic determinants free to bind with the anti-HLA antibodies possibly existing in recipient's serum in the subsequent reaction; by which the fixed anti-HLA antibodies are able to capture all corresponding HLA molecules (all HLA class I or II molecules, or both molecules) and do not interfere with the recipient's HLA antibodies binding to the same HLA molecule in different antigen determinants. The solid carries can be any kin of microparticles or microspheres made from any materials. If the solid carriers are magnetic beads, the captured HLA molecules on the surfaces of microparticles can be separated by magnetic field. If the solid carriers are synthetic microparticles, then centrifugation can be used for the same purpose.

In a particular donor specific HLA crossmatch, when the donor's cell lysate is incubated with the above microparticles, the anti-HLA antibodies on the surface of microparticles will efficiently capture the HLA antigens to form anti-HLA-HLA molecules complexes. After washes, the above microsparticls will be further incubated with the serum of a graft recipient. The HLA antibodies existed in the recipient's serum, if there is any, will bind to the above captured donor specific HLA molecules (HLA antigens) and fix recipient's autologous complements. After further washes, fluorescent anti-human IgG or anti-human complements antibodies will be added to the reaction and fluorescence signals on the microparticles can be detected by a flow cytometer. By using this system, donor specific HLA antibodies (IgG) or donor specific HLA complement fixing antibodies in a recipient serum could be either directly or indirectly identified.

The fluorescence labels used in the method of the invention can be any fluorescein or fluorescent protein that can be measured by a flow cytometer, such as FITC, PE, PE-Cy5, Cy5, Per-CP, Cy7 etc. Fluorescence can be directly conjugated with complements, anti-complement antibodies or antibodies against the particular antigen on surface of target cells according to conventional means of the art.

In the method of the invention, the solid carrier used to be fixed with antigen or antibody can be any solid carrier conventionally used in the art, such as magnetic beads, particles or microtiter-plates.

In another aspect, the invention relates to a kit for implementing the method of the invention, which comprises at least containers containing a fluorescence-labeled complement or fluorescence-labeled anti-complement antibody and a suitable buffer respectively.

In a preferred embodiment of the invention, said kit further comprises an antibody against the antigens on the surface of target cells for identifying cell populations, which is fluorescence labeled, or coated on solid carriers such as magnetic beads or microtiter-plates, or coated on micro-particles, or coated on fluorescence-labeled particle.

In a preferred embodiment of the invention, said kit further comprises an anti-HLA antibody directed against the constant region of HLA antigens, which is able to capture the donor's HLA molecules.

Because the method of the invention uses fluorescence as a tracer, it has a high sensitivity.

As the detection target is HLA-CFAbs-complement complex that is generated in the initial stage of CDC effects when the method is applied to crossmatch, the reaction time is much less compared with the existing methods.

As the method of the invention can detect the autologuous complements in serum of the recipient and the main components of the reaction system are serum from the recipient and cells or cell lysates from the donor, the reaction conditions of the method is most close to or the best mimic of the physiological internal environment conditions of the recipient.

Because binding activity of the CFAbs and complements in serum can be kept at low temperature for a long time (several years), the method of the invention has stability and good reproducibility in methodology.

Because flow cytometry is used for fluorescence measuring and analyzing instrument, CDC effects on a particular cell population can be semi-quantitatively determined with a single or/and multiple fluorescence labels, and CDC effects on different cell populations can be simultaneously determined in a same reaction tube. The collected data can be statistically analyzed to obtain scientific, accurate and reliable results. The experimental data can be permanently stored in a computer.

A comparison of Terasaki's microlymphocytotoxicity test (Terasaki's CDC), IgG-FXM, and the method of the invention (CDC-FXM) can be made as follows.

TABLE 1
Comparison of the three cross-match methods

| | Method | | |
|---|---|---|---|
| Index | Terasaki's CDC | IgG-FXM | CDC-FXM |
| Antibody Detected | CFAbs | Non-CFAbs | CFAbs |
| | | and CFAbs | |
| Result Reading | Cell death rate | IgG | CFAbs binding |
| | (CDC final | binding rate | rate (CDC |
| | effect) | | initial effect) |
| Complement | From animal | Not involved | Autologous |
| | (rabbit) | | complements in |
| | | | serum of recipient |
| Close to In Vivo | Not close | Close | Most close |
| Conditions of the | | | |
| recipient | | | |
| Active cell | >85% | ≧50% | ≧50% |
| Sensitivity | Low | High | High |
| Specificity | Good | Not good | Good |
| Time | Long | Shorter | Shortest |
| | (>3 hours) | (about 2 hours) | (˜40 minutes) |
| Stability | Not good | Good | Good |
| Reproducibility | Not good | Good | Good |
| False positive | High | High | Lowest |
| False negative | High | Low | Low |
| Cell pre-separation | Needed | Not needed | Not need |
| in cross-match of | | (identified by | (identified by |
| T and B cells | | fluorescence label) | fluorescence label) |
| Sample from patient | Suitable | Not suitable | Suitable |
| treated by IVIG | | (false positive) | |
| Personal | Dependent | not dependent | not dependent |
| Experience | | | |
| Dependent | | | |

The CFAbs cross-match method established by the invention has many advantages over the existing cross-match methods. It is rational in theory, and leads to the results completely fitting the theoretical basis.

### BRIEF DESCRIPTION OF THE DRAWINGS

FIG. 1 shows the result of T lymphocyte cross-match. Single side blind cross-match results of 117 cases of T lymphocyte cross-match by two methods are showed. 109 cases were coincident by the traditional CDC serological method and the method of the invention. Among these cases, 26 were positive (A), 83 were negative (B). 8 cases were negative by traditional CDC serological method, but positive by the method of the invention (C). These results proved that the rate of false negative in traditional CDC serological method is up to 7%.

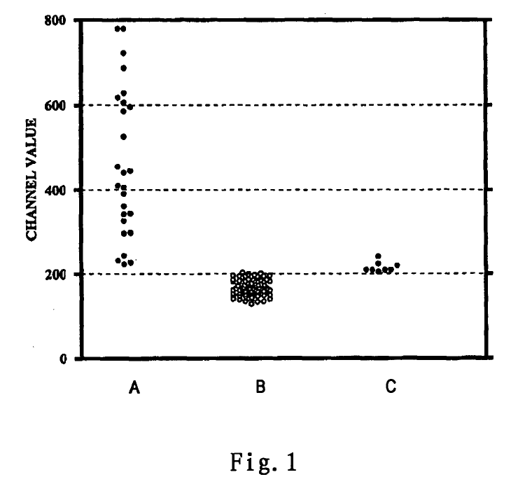

FIG. 2 shows the results of complement C1q spiking experiment. Serum containing CFAbs was diluted in 1:4 with a diluting buffer. Complements in the serum were inactivated at 56° C. for 30 minutes. Human C1q in different amounts were added into the inactivated serum to determine the necessary C1q concentration for a positive result by the method of the invention. The results showed that the lowest C1q necessary concentration for a positive crossmatch was 3 μg/ml (average C1q concentration in normal human is 75 μg/ml).

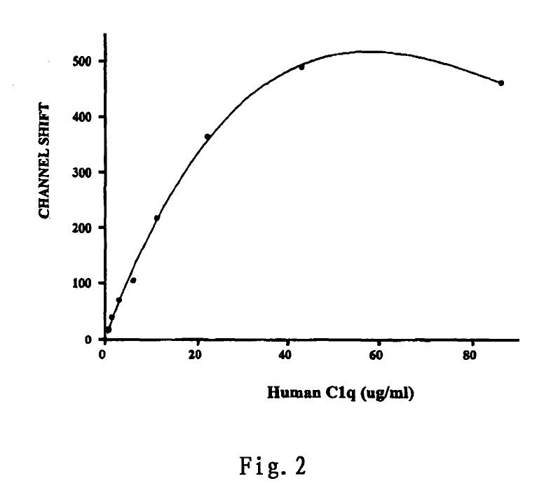

FIG. 3 shows the results of CFAbs spiking experiment. Total IgG was purified from the serum containing CFAbs by Protein-G affinity chromatography. Different amounts of the purified IgG were added back to the serum to determine the necessary IgG concentration for a positive crossmatch by the method of the invention.

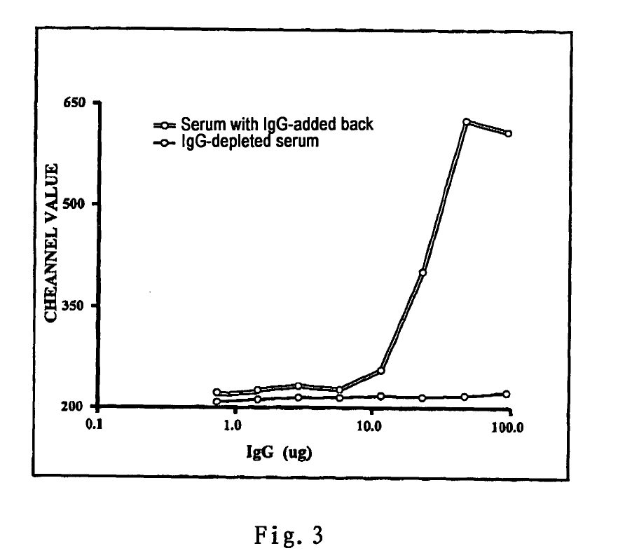

The results led to the following conclusions:

1. When IgGs was depleted from CFAbs positive serum, complements of the serum couldn't be fixed onto the cell surface. It means that complement fixing rate, which is detected by the method of the invention, directly reflects the binding rate of CFAbs on the surface of target cells and has positive correlation with the latter.

2. The lowest necessary IgG concentration for a positive crossmatch by the method of the invention was 0.4 mg/ml (average IgG concentration of normal human serum is 12.5 mg/ml).

FIG. 4 shows the binding between Intravenous Immunoglobulin Gamma (IVIG) and purified HLA antigen. Purified HLA antigen coated on plastic beads was reacted with different amount of IVIG The amount of IVIG bound to HLA antigen was determined by fluorescence labeled antibody against human IgG on a flow cytometer. The result showed that IVIG in the concentration far below the average physiological level (<10 mg/ml) could give a significant binding to purified HLA antigens.

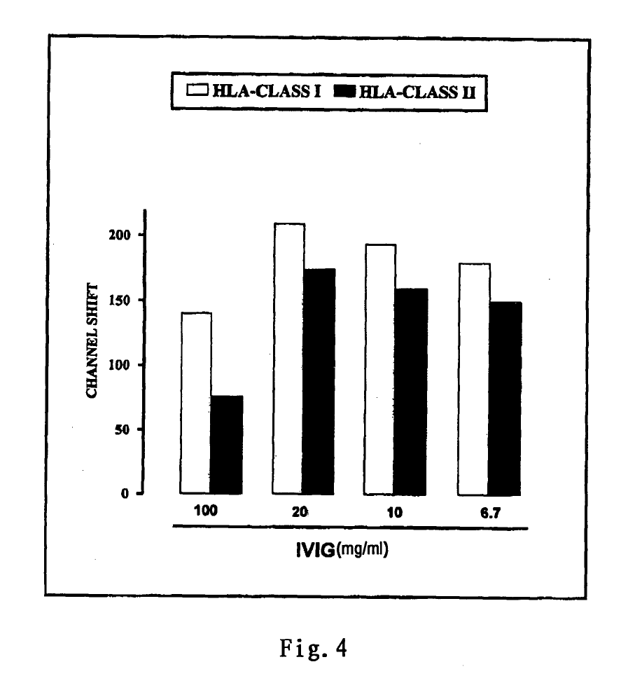

FIG. 5 shows the cross-match results of a patient before and after IVIG treatment. The same recipient was cross matched by the method of the invention, IgG-FXM and CDC serological method respectively.

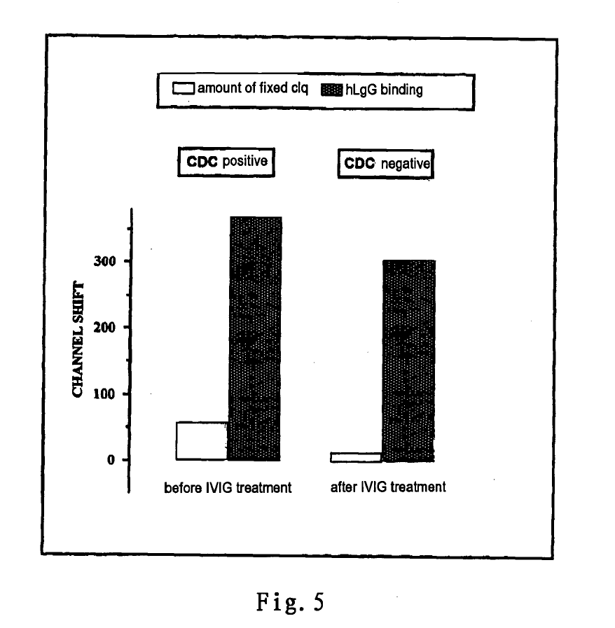

The positive cross-match results were seen in all three methods before IVIG treatment. The immunological rejection response of the recipient was inhibited after IVIG treatment with an improved clinical outcome. After IVIG treatment, positive cross-matches turned to negatives by the method of the invention and CDC serologic method, consistent with the clinical outcome; but still positive by IgG-FXM method.

FIG. 6 shows that IVIG inhibits the complement fixation by CFAbs. Different amounts of IVIG were added into a serum containing CFAbs, and reacted with the corresponding target cells. The amount of complement fixed on target cells and IgG binding were parallelly determined by a flow cytometer. The results showed that IVIG strongly inhibited the complement fixation ability of CFAbs, but had no obvious influence on IgG bindings. The results were consistent with the clinical effect of the IVIG treatment, which can effectively inhibit graft rejection in transplantation. IVIG inhibits CDC effects in graft rejection by binding to the surfaces of target cells to block the binding sites of CFAbs.

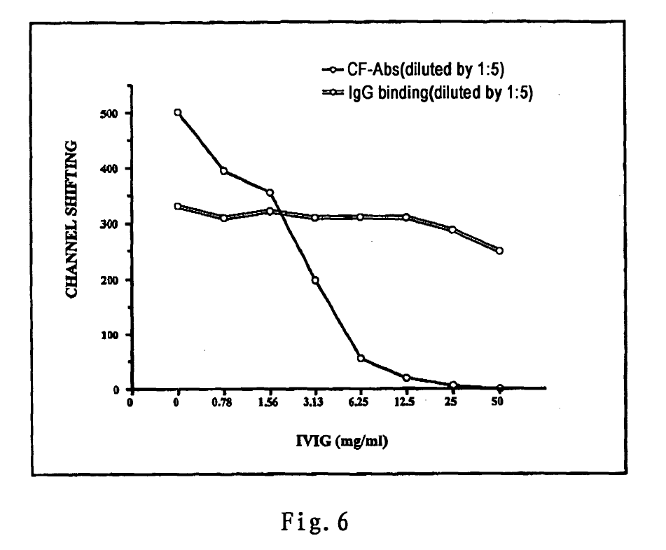

FIG. 7 shows the binding of normal human IgG with HLA antigen. IgG from normal female (A) who had been pregnant for more than 3 times; and normal young male (B) who had never received blood transfusion and transplantation, were purified by Protein-G affinity chromatography. The purified IgG was then reacted with purified HLA antigen coated on solid particles. The binding of IgG with HLA antigen was determined by fluorescence labeled antibody against human IgG on a flow cytometer.

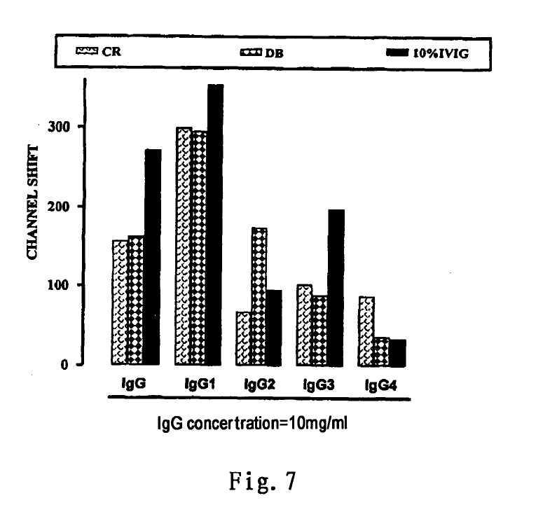

The results showed that the purifed IgG from either A or B was able to significantly bind with HLA antigens. This finding further proved that there are natural HLA antibodies existed in normal person who has never been immunologically sensitized with any foreign HLA antigens.

### BEST MODE FOR CARRYING OUT THE INVENTION

### EXAMPLE 1

### T Cell Crossmatch Method

(1) To a 1.5 ml Eppendorf centrifuge vial, 0.1˜0.25×10⁶ mononuclear cells of the donor prepared by Ficoll-Hypaque density-gradient centrifugation method was added.

(2) The cells were centrifuged at 800 g for 5 minutes and the supernatant was aspirated.

(3) 25 ul test serum of the recipient was added to the vial and mixed well genteelly. Then the mixture was incubated for 10 minutes at room temperature (˜25° C.).

(4) 30 ul FITC-anti human C1q (BIODESIGN; 1:30 diluted in 5% FCS/HBSS) and 10 ul PerCP CD3 (BD Biosciences) were added and mixed well. The mixture was incubated for an additional 20 minutes at 4° C.

(5) 0.5 ml wash buffer (5% FCS/HBSS) was added to the reaction mixture. The mixture was centrifuged and the supernatant was aspirated. The washing was repeated once again.

(6) After the second wash, the cell pellet was resuspended in 0.5 ml 1% paraformaldehyde. The samples were analyzed on a flow cytometer (FACScan; BD Biosciences) by collecting 5,000 cells for each sample.

### EXAMPLE 2

### Direct Cross-Match Method

(1) The serum of the recipient was incubated at 56° C. for 30 minutes to inactivate autologous complements.

(2) 25 ul of the above serum and 10 ul of FITC-C1q were added to a test tube containing 0.1˜0.25×10⁶ mononuclear cells from the donor, and the mixture was incubated at room temperature for 10 minutes.

(3) 10 ul PerCP CD3 (BD Biosciences) was added to the tube and mixed well. The mixture was incubated for an additional 20 minutes at 4° C.

(4) The samples were washed and analyzed according to the same process as in the steps (5) and (6) in Example 1

### EXAMPLE 3

### B Lymphocyte Cross-Match

The substantially same procedures as in Example 1 were carried out except that PerCP CD3 was replaced with PE labeled CD19.

### EXAMPLE 4

### Determination of CFAbs Against Vascular Endothelial Cells

(1) 25 ul serum of the recipient and 10 ul of FITC-C1q or FITC-C3 were added into a test tube containing 0.1˜0.25×10⁶ pooled human vascular endothelial cells (for example cultured cell lines of human vascular endothelial).

(2) The mixture was incubated at room temperature for 30 minutes.

(3) The same procedures as in steps (5) to (6) of Example 1 were carried out.

### EXAMPLE 5

### Donor Specific HLA Class I Antigen Crossmatch Method

(1). 1˜5×10⁶ donor's nucleate cells was added to a test vial containing 0.5 ml lysis buffer and 2×10⁶ microparticles preconjugated with anti-HLA antigen I antibodies.

(2). The mixture was incubated at room temperature or 37° C. for 30 minutes. 1 ml wash buffer (0.1% Toween-20; 2% BSA in PBS) was added to the vial and the mixture was centrifuged at 3000 g in an Eppendorf centrifuge for 5 minutes.

(3). The pellet was washed once again and the supernatant was removed. 25 ul serum of the recipient was added to resuspend the microparticles. The mixture was further incubated at room temperature for 10 minutes.

(4). 50 ul FITC-anti C1q or FITC-anti C3 antibodies were added and mixed well. The mixture was incubated at room temperature or 37° C. for an additional 30 minutes.

(5). The microparticles were washed twice with 1 ml wash buffer.

(6). The microparticles were resuspended in 0.5 ml PBS and the fluorescence signals were measured on a flow cytometer.

(7). The fluorescent density above negative AB serum control will be considered as a positive crossmatch.

### EXAMPLE 6

### Donor Specific HLA Class II Antigen Crossmatch Method

The substantially same procedure as Example 5 was carried out except replacing the microparticles preconjugated with anti-HLA antigen I antibodies with microparticles preconjugated with anti-HLA antigen II antibodies in step 1.

### EXAMPLE 7

### Donor Specific HLA Class I/II Antigens Crossmatch Method

The substantially same procedure as Example 5 was carried out except using an equal number of microparticles preconjugated with anti-HLA antigen I antibodies and microparticles preconjugated with anti-HLA antigen II antibodies in step 1.

### EXAMPLE 8

### Reaction Conditions and Kit Components

(1) Incubation: the temperature was controlled between 1° C. to 50° C., generally at 4° C., room temperature (25° C.±5° C.) or 37° C. The duration of incubation was 5 to 60 minutes. CFAbs bind to the corresponding antigens during incubation. If the sample is a serum, the complements therein could be selectively fixed onto the CFAbs-Ag complexes of the cell surfaces.

(2) Washing: washing solution could be any physiological buffer that has no impacts on cell shapes, cells activity, and CFAbs-Ags-labeled complements. The washes could remove the free proteins in liquid phase, especially unbound free antibodies, soluble antigen-antibody complexes, and complements etc. After washing, only the cells with CFAbs-Ags-labeled complements (when cells used for targets) were left in the reaction system.

### EXAMPLE 9

### Negative Serum Control, Positive Serum Control, and Self Control Samples Set Up

(1) Negative Serum Control Setting:

Pooled serum of males with AB blood type could be used for negative controls. This control serum should be tested to ensure not containing any CFAbs. Complements in the serum could be optionally inactivated at 56° C. for 30 minutes.

Cells could be either from the same group, from which the serum was obtained, or from different normal persons. The number of cell samples must be more than 10. Mean value (X) and standard deviation (SD) of the fluorescence intensity, which was obtained from the above samples by cross-match experiment, were calculated. X+2SD was taken as upper limit to judge negative result. Results higher than X+2SD was judged as cross-match positive. Results lower than X+2SD was judged as cross-match negative.

(2) Positive Serum Control Setting:

Positive serum control could be prepared from a pooled serum with a high PRA (>50%) to ensure to covere most HLA antigens (>95% HLA antigens). Three different CFAbs-positive quality controls were prepared by preliminary tests, i.e. serum samples containing CFAbs in a high, middle, and low concentration respectively. The sample containing CFAbs of a low concentration was set to be about 50 Channel Shifts.

(3) Self Control Setting:

Autologous serum and mononuclear cells from the recipient will be used for a self control reaction. The result of the self control sample will be compared with the negative control mentioned above to determine the existence of autologous CFAbs in the recipient with some autoimmune disease.

### EXAMPLE 10

### Microparticles Preconjugated with Anti HLA Class I and Class II Antibodies

The mcroparticles can be used for capturing pure HLA molecules from a donor to detect the corresponding HLA antibodies from a recipient (Donor specific HLA antigen crossmatch).

### EXAMPLE 11

### Fluorescent Antibodies

Fluorescent antibodies can be selected from the group consisting of anti-C1q, anti-C3; antiCD2, antiCD3, antiCD4, antiCD8, antiCD16, antiCD19, antiCD20, antiCD45, and antiCD56 antibodies. The fluorescent labels of above antibodies can be selected from the group consisting of FITC, PE, PerCP, Cy5, PE-Cy5, Cy7 etc.

### EXAMPLE 12

### Identification of CFAbs Type

Following steps can be carried out for identifying the type of CFAbs in cross-match positive serum samples: treating the serum with DTT (decomposing IgM), and then performing cross-match again. If the result turns to negative, the type of CFAbs in the positive sample is IgM. Otherwise, the type of CFAbs in the positive sample is IgG.

### EXAMPLE 13

### Specificity and Quality Control

The specificity of the flow cytometry crossmatch method of the invention was evaluated. The results were shown in the following table 2.

TABLE 2
Test results of specificity of the method of the invention

| | Target | | | | | | | | |
|---|---|---|---|---|---|---|---|---|---|
| CELL | CELL | Donor-1 | Donor-2 | Donor-3 | Donor-4 | Donor-5 | Donor-6 | Donor-7 | Donor-8 |
| ID | TYPE | B51+ | B51+ | A11+ | A11+ | A2+ | A2+ | A2+ | A2+ |
| 06FP82119 | A2+; A11+ | 19 | 13 | 287 | 137 | 593 | 269 | 232 | 672 |
| LF28291 | A11+ | 8 | 16 | 217 | 187 | 7 | 7 | 8 | 7 |
| K16297 | A2+ | 3 | 19 | 24 | 1 | 578 | 306 | 124 | 666 |
| K16355 | A2+; B51+ | 662 | 302 | 20 | 9 | 660 | 492 | 374 | 549 |
| M17563 | A2+; B52+ | 692 | 301 | 7 | 4 | 544 | 290 | 68 | 520 |

The inter-assay/intra-assay variations were measured with regard to the cytometry crossmatch method of the invention. The results were shown in the following table 3.

TABLE 3
Quality Control results

| | | CV % | | |
|---|---|---|---|---|
| QC | N | QC-L | QC-M | QC-H |
| Inter-Assay | 10 | 1.88 | 3.61 | 4.2 |
| Intra-Assay | 17 | 4.6 | 6.2 | 3 |

1. A method of crossmatch by determining complement-fixing antibodies, comprising a step of contacting antigens from the donor with a sample from the recipient potentially containing complement-fixing antibodies against the antigens and related complements, a step of contacting a labeled complement or labeled anti-complement antibody, a step of removing the non-bound labels from the reaction system, and a step of detecting the signals of the label or reaction signals to determine the presence of said complement-fixing antibodies in the sample; wherein said antigens from the donor are whole nucleate cells or donor's HLA molecules pre-captured by anti-HLA antibodies on the surfaces of solid carriers.

2. The method according to claim 1, wherein said label is a fluorescence label and the fluorescence intensity of the reaction product is determined by a flow cytometer.

3. The method according to claim 1, wherein said whole cells are T lymphocytes and/or B lymphocytes.

4. The method according to claim 1, wherein said anti-HLA antibodies are anti-HLA class I or/and anti-HLA class II antibodies.

5. The method according to claim 1, wherein said anti-HLA antibodies are fixed on solid microparticles made from any materials.

6. The method according to claim 1, wherein said a sample is a body fluid derived from a human being or animal.

7. The method according to claim 6, wherein said body fluid is serum, plasma, cerebrospinal fluid, spinal fluid, amniotic fluid, salvia, thorax fluid or abdominal cavity fluid.

8. The method according to claim 1, wherein said complement is selected from the group consisting of C1(C1q, C1r, C1s), C2, C3, C4, C5, C6, C7, C8, C9, C1qrs, C1qrs, C2a, C2b, C3a, C3b, C4a, C4b, C4d, C4b2, iC3b, C4b2b, C4b2b3b, C3bBb, C3bnBb, C5a, C5b, C5b67, C5b˜8, C5b˜9, C1-inhibitory factor, C4-binding protein, D factor, B factor, P factor (properdin), I-factor, H-factor, S-protein, Ba, Bb, MBP, MCP, DAF(CD55), CR1, CR2, CR3, CR4, CR5, C3aR, C2aR, C4aR, C1qR and CD59.

9. The method according to claim 8, wherein said complement is C1q, C3 or C4d.

10. The method according to claim 1, wherein said labeled complement is derived form human being, animal, chemical synthesis or genetic engineering.

11. The method according to claim 1, wherein the complements contained in the sample from the recipient can be directly used for the testing, or inactivated before the testing.

12. The method according to claim 1, wherein said anti-complement antibody is a polyclonal antibody, monoclonal antibody, chimeric antibody, single-chain antibody or an antibody fragment.

13. The method according to claim 2, wherein said fluorescence label is selected from the group consisting of FITC, PE, Cy5, PE-Cy5, Per-Cp and Cy7.

14. The method according to claim 1, further comprising adding at least one antibody against a particular antigen on the surface of the cells in order to identify a particular cell population and determine the complement-fixing antibodies on the surfaces of the cell populations.

15. The method according to claim 14, wherein said antibody is specifically against any CD antigen of nucleate cells.

16. The method according to claim 15, wherein said antibody is specifically against a CD antigen of T lymphocytes or B lymphocytes.

17. A kit for implementing the method of claim 1, comprising at least containers containing a fluorescence-labeled complement or fluorescence-labeled anti-complement antibody and a suitable buffer respectively.

18. The kit according to claim 17, which further comprises anti-HLA class I or class II antibodies or a mixture of above two antibodies.

19. The kit according to claim 18, wherein said anti-HLA class I or class II antibodies are conjugated or coated on solid microparticles made from any materials; therefore being able to capture corresponding HLA antigens from the donor's cell lysates.

20. The kit according to claim 17, further comprising at least one antibody against a particular antigen on the surface of the cells for identifying a particular cell population and determining the complement-fixing antibodies on the surfaces of the cell populations.
